(19) United States
(12) Patent Application Publication
(10) Pub. No.: US 2003/0065301 A1
(43) Pub. Date: Apr. 3, 2003

(54) Disposable diaper and wipe combination

(76) Inventors: Michelle Elliott, Jamaica, NY (US); Dennis Ronald Dunston, Alexandria, VA (US)

Correspondence Address: MICHELLE ELLIOTT, 168-14 144TH ROAD, JAMAICA, NY, 11434, US

(21) Appl. No.: 09/967,799

(22) Filed: Oct. 1, 2001

(51) Int. Cl. A61F 13/15; A61F 13/20

(52) U.S. Cl. 604/385.06

(57) ABSTRACT

A disposable diaper that contains or has attached wipe(s). The wipe(s) will be one or more individual pieces of wet/dry/fragrance/lotion material. Material means (cloth, tissue, napkin, towel, towelette, sponge, handi-wipe).

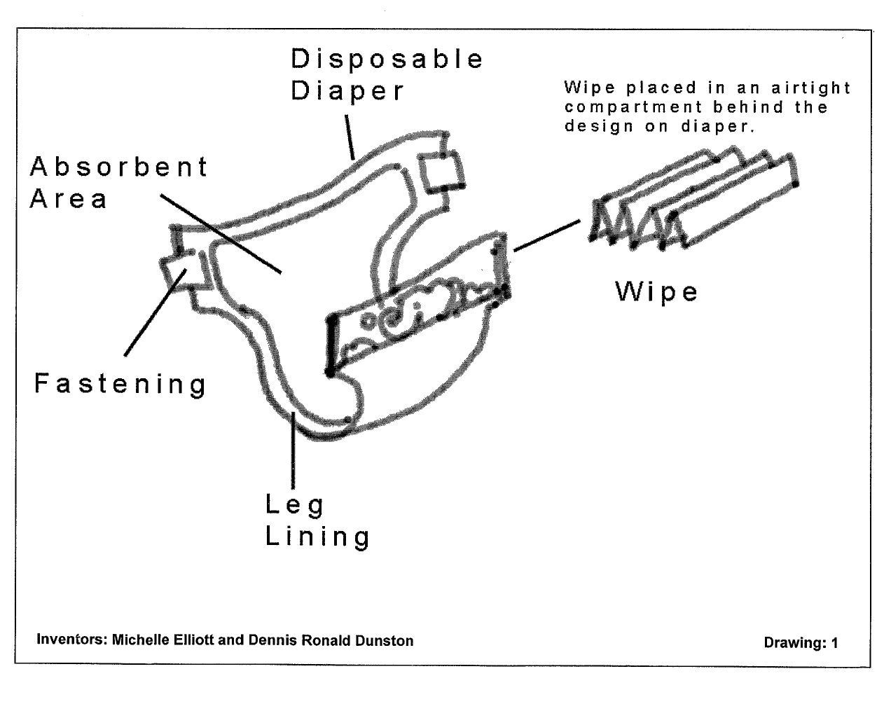

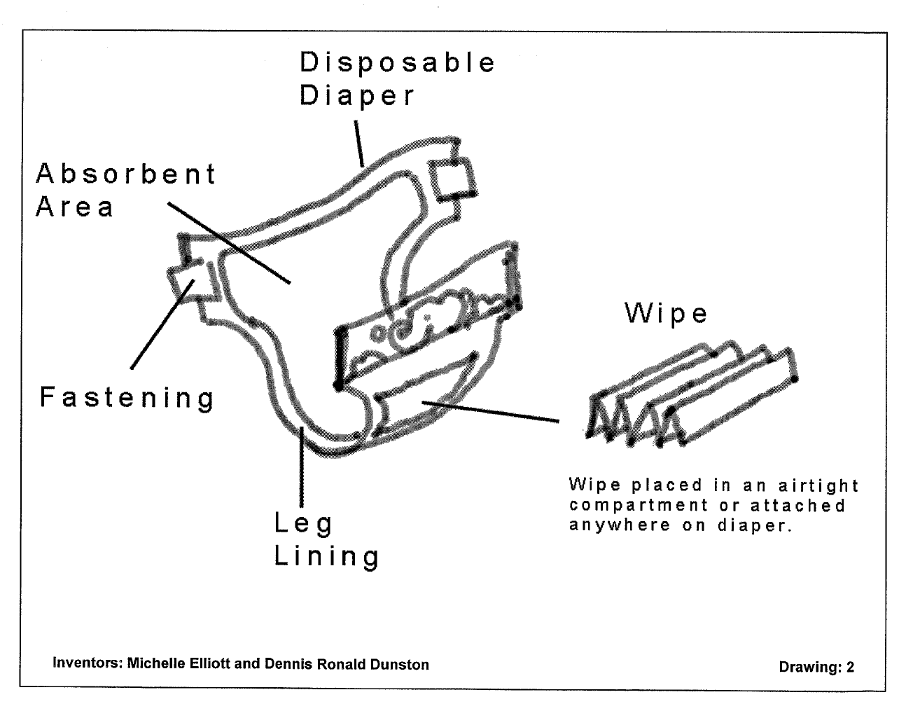

### 2. CROSS REFERENCE TO RELATED APPLICATIONS

None

### 3. STATEMENT REGARDING FED SPONSORED R & D

None

### 4. REFERENCE TO SEQUENCE LISTING, A TABLE, OR A COMPUTER PROGRAM LISTING APPENDIX

None

### 5. BACKGROUND OF THE INVENTION

This invention relates to prior art “The Disposable Diaper”. This invention increases the functionality of the prior art by containing or attaching a fresh wipe(s). This invention combines the diaper and the wipe(s) eliminating the need to carry two separated objects or products.

This invention will provide a fast, easy and convenient way to save time for baby boomers and parents on the go with infant/toddlers to quickly change a diaper by combining the two products (diapers and wipes). Diapers on the go! Most customers (parents, schools, day care facilities, day care providers, baby-sitters) run out of wipes or have to carry/buy the wipes in the bulky container and sometimes they forget to bring the wipes with them. By combining the two products (diapers and wipes), the consumer will never forget, lose, run out of or have to lug around the bulky wipe container.

### 6. BRIEF SUMMARY OF THE INVENTION

This invention will provide a fast, easy and convenient way to save time for baby boomers and parents on the go with infant/toddlers to quickly change a diaper by combining the two products (diapers and wipes). Diapers on the go! Most customers (parents, schools, day care facilities, day care providers, baby-sitters) run out of wipes or have to carry/buy the wipes in the bulky container and sometimes they forget to bring the wipes with them. By combining the two products (diapers and wipes), the consumer will never forget, lose, run out of or have to lug around the bulky wipe container.

### 7. BRIEF DESCRIPTION OF THE DRAWINGS

There are two drawings of this invention. This invention builds on the prior art in the following ways. (1) The wipe placed in an airtight compartment behind the design on diaper. (2)The wipe placed in an airtight compartment or attached anywhere on diaper.

### 8. DETAILED DESCRIPTION

The disposable diaper, which will contain or have attached wipe(s). The diaper container or attachment will be a closable/reclosable, storage container/pouch/compartment/pocket. The wipe(s) will be individual piece(s) of wet/dry/fragrance/lotion material. Material means (cloth, tissue, napkin, towel, towelette, sponge, handi-wipe).

What is claimed is:

1. A disposable diaper, which contains or has attached wipe(s). The wipe(s) will be one or more individual pieces of wet/dry/fragrance/lotion material. Material means (cloth, tissue, napkin, towel, towelette, sponge, handi-wipe).
